(19) United States
(12) Patent Application Publication
(10) Pub. No.: US 2013/0144317 A1
(43) Pub. Date: Jun. 6, 2013

(54) Position feedback control for a vitrectomy probe

(71) Applicant: Salomon Valencia, Aliso Viejo, CA (US)

(72) Inventor: Salomon Valencia, Aliso Viejo, CA (US)

(21) Appl. No.: 13/308,995

(22) Filed: Dec. 1, 2011

(51) Int. Cl. A61B 17/32

(52) U.S. Cl. 606/170

(57) ABSTRACT

A method of controlling a surgical system using position feedback control, includes selectively operating a cutter having a cutting mechanism, with the cutting mechanism having an inner cutting tube and an outer cutting tube. The outer cutting tube has a tissue-receiving port formed therein, and the inner cutting tube has a cutting edge axially displaceable relative the tissue receiving port to cut tissue therein. The method also includes sensing the displacement of the inner cutting tube relative to the outer cutting tube with a sensor and changing operational timing of a probe driver based on the displacement sensed by the sensor.

### BACKGROUND OF THE INVENTION

The present invention pertains to vitrectomy probe systems. More particularly, but not by way of limitation, the present invention pertains to position feedback control for a cutter on a vitrectomy probe.

Microsurgical procedures frequently require precision cutting and/or removing various body tissues. For example, certain ophthalmic surgical procedures require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous must be done with great care to avoid traction on the retina, the separation of the retina from the choroid, a retinal tear, or, in the worst case, cutting and removal of the retina itself. In particular, delicate operations such as mobile tissue management (e.g. cutting and removal of vitreous near a detached portion of the retina or a retinal tear), vitreous base dissection, and cutting and removal of membranes are particularly difficult.

The use of microsurgical cutting probes in posterior segment ophthalmic surgery is well known. These cutting probes typically include a hollow outer cutting member, a hollow inner cutting member arranged coaxially with and movably disposed within the hollow outer cutting member, and a port extending radially through the outer cutting member near the distal end thereof. Vitreous humor and/or membranes are aspirated into the open port, and the inner member is actuated, closing the port. Upon the closing of the port, cutting surfaces on both the inner and outer cutting members cooperate to cut the vitreous and/or membranes, and the cut tissue is then aspirated away through the inner cutting member.

Variations in characteristics of cutter components, including those from initial critical component tolerances, can introduce inconsistencies in operation and can restrict maximum cut rate potential across vitrectomy probes. To address these variations, current systems are operated according to parameters suitable for a large population of probes, instead of operated according to parameters ideal for a single particular probe. For example, long operational time periods that the valve is on and off (pulse width of the valve) are selected so that there is sufficient pressure to close and open a population of probes rather than to specify the necessary pulse pressure to satisfy a particular system. For moderate cut rate applications (i.e. 7500 cpm), specifying the pulse width of the valve signal according to a large population of probes may be suitable. However, as cut rates increase, specifying the appropriate valve timing sequence becomes more challenging because the periods of cycle become smaller with higher cut rates. Any added margin to the design to ensure the probe closes and opens now begins to restrict the ability to maximize cut rate.

Accordingly, what is needed is an ability to send a prescribed control signal to the probe to achieve a desired response. This may maximize the performance of the system (cut rate & duty cycle) and minimize the effect of tolerances in the system.

The present disclosure is directed to addressing one or more of the deficiencies in the prior art.

### SUMMARY OF THE INVENTION

In one exemplary aspect, the present disclosure is directed to a surgical system having position feedback control. The system may include a probe driver and a vitrectomy probe having a cutting mechanism. The cutting mechanism may include an inner cutting tube and an outer cutting tube, with the outer cutting tube having a tissue-receiving port formed therein. The inner cutting tube may have a cutting edge axially displaceable relative the tissue receiving port to cut tissue therein. A sensor may be disposed and configured to detect the displacement of the inner cutting tube relative to the outer cutting tube and communicate a signal indicative of the relative displacement of the inner cutting tube. A controller may be in communication with the sensor and with the probe driver. The controller may be configured to change operational timing of the probe driver based on the signal communicated from the sensor.

In one aspect, the controller is configured to determine a desired stroke for the inner cutting tube based on the signal communicated from the sensor. In another aspect, the controller is configured to compare the sensed relative displacement of the inner cutting tube to a desired displacement and modify the operational timing of the valve based on the difference between the sensed relative displacement and the desired displacement.

In another exemplary aspect, the present disclosure is directed to a method of controlling a surgical system using position feedback control. The method may include the steps of selectively operating a cutter having a cutting mechanism, the cutting mechanism having an inner cutting tube and an outer cutting tube, with the outer cutting tube having a tissue-receiving port formed therein. The inner cutting tube may have a cutting edge axially displaceable relative the tissue receiving port to cut tissue therein. The method may also include the steps of sensing the displacement of the inner cutting tube relative to the outer cutting tube with a sensor and changing operational timing of a probe driver based on the displacement sensed by the sensor.

In another exemplary aspect, the present disclosure is directed to a method of using position feedback control to control a cutting mechanism of a vitrectomy probe having an inner cutting tube and an outer cutting tube, the outer cutting tube having a tissue-receiving port formed therein the inner cutting tube having a cutting edge axially displaceable relative the tissue receiving port to cut tissue therein. The method may include the steps of setting up the vitrectomy probe to detect a port just open position and a port just closed position, identifying a target stroke length from the port just open position to the port just fully closed position, receiving an input from a health care provider during a surgical procedure, sensing an actual stroke length during the surgical procedure with a sensor, comparing the actual stroke length to the target stroke length, and adjusting the system to change the actual stroke length to more closely match the target stroke length.

It is to be understood that both the foregoing general description and the following detailed description are exemplary and explanatory only and are intended to provide further explanation of the invention as claimed. The following description, as well as the practice of the invention, sets forth and suggests additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is an illustration of an exemplary surgical machine according to one aspect of the present disclosure implementing the principles and methods described herein.

FIG. 2 is a diagram of an exemplary system on the surgical machine with feedback control according to one aspect of the disclosure.

FIG. 3 is an illustration of an exemplary vitrectomy probe in cross-section operable in accordance with the principles and methods described herein.

FIG. 4 is an illustration of a sectional view of a distal end of a cutter of the probe of FIG. 3.

FIG. 5 is an illustration of an exemplary wave form identifying a detected displacement of an inner cutter member relative to an outer cutting member of a vitrectomy probe in accordance with one aspect of the present disclosure.

FIGS. 6 and 7 are illustrations of exemplary wave forms identifying a detected displacement of an inner cutter member relative to an outer cutting member of a vitrectomy probe at an 80% probe duty cycle and at a 20% probe duty cycle, respectively.

FIG. 8 is an illustration of a flow chart showing exemplary setup steps in accordance with one aspect of the present disclosure.

FIG. 9 is an illustration of an exemplary flow chart showing a method for controlling stroke using position feedback control in accordance with one exemplary aspect of the present disclosure.

### DETAILED DESCRIPTION OF THE PREFERRED EMBODIMENTS

Reference is now made in detail to exemplary embodiments of the invention, examples of which are illustrated in the accompanying drawings. Wherever possible, the same reference numbers are used throughout the drawings to refer to the same or like parts.

The present disclosure is directed to a surgical system including a vitrectomy probe for performing ophthalmic surgeries. The surgical system is arranged and configured to use position feedback control to track the probe's cutter movement and to optimize control of the vitrectomy probe. A sensor detects the position of the vitrectomy probe's cutter and a closed loop control system uses the position of the cutter to generate a specific response to the cutter. In one example, the signal received from the sensor is used to track the reciprocating position of the cutter with respect to time. It may be a sinusoidal type of waveform, where the high peak amplitude of the signal indicates how far the cutter traveled to its open position. Similarly, the low peak amplitude of the signal indicates how far the cutter has traveled to its closed position. The peak to peak amplitude differential represents the stroke of the cutter. A saturated peak amplitude indicates that the cutter is idle or stopped at its maximum closed or minimum open positions. The difference between the maximum closed or maximum open position indicates the cutter's maximum stroke.

By obtaining data regarding characteristics of the cutter operation in a closed loop manner, the system can operate the cutter to maximize the full performance of the system and increase the robustness of the design. For example, system tolerances can be minimized, making the components easier to create while obtaining similar or better performance. This can lead to a more cost efficient design. In addition, higher cut rates can be achieved to increase the dynamic range of operation and address specific aspects of the vitrectomy surgery (i.e. core vitrectomy, membrane dissection, etc.). Furthermore, variable port duty cycle can be achieved to increase the dynamic flow range at a particular cut rate. In addition, variable port aperture can also be achieved to reduce flow without changing vacuum and/or selectively aspirate specific particle size tissue into the probe.

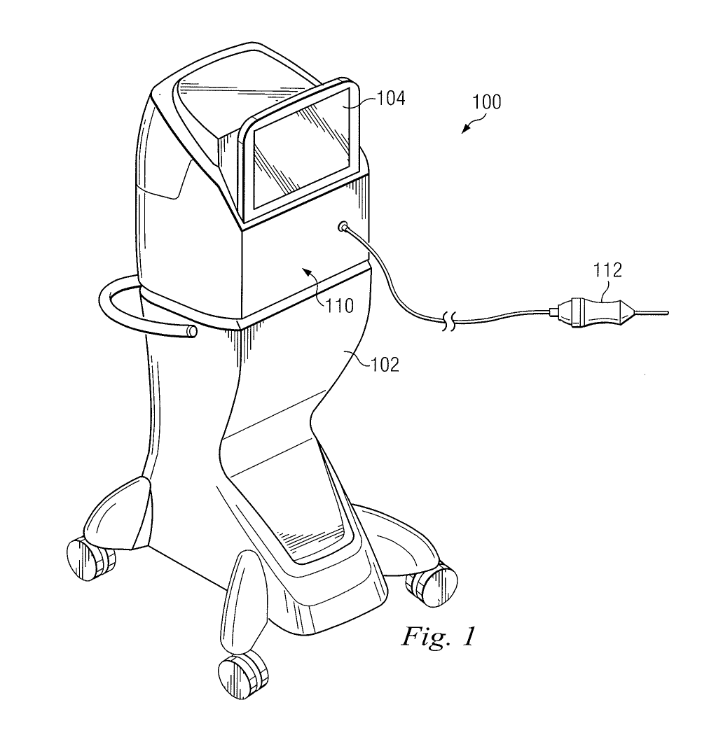

FIG. 1 illustrates a vitrectomy surgical machine, generally designated 100, according to an exemplary embodiment. The machine 100 includes a base housing 102 and an associated display screen 104 showing data relating to system operation and performance during a vitrectomy surgical procedure. The machine includes a vitrectomy probe system 110 that includes a vitrectomy probe 112 and is configured to provide position feedback control to compensate for variations in operation due to mechanical inconsistencies created by tolerances, component wear, or other factors.

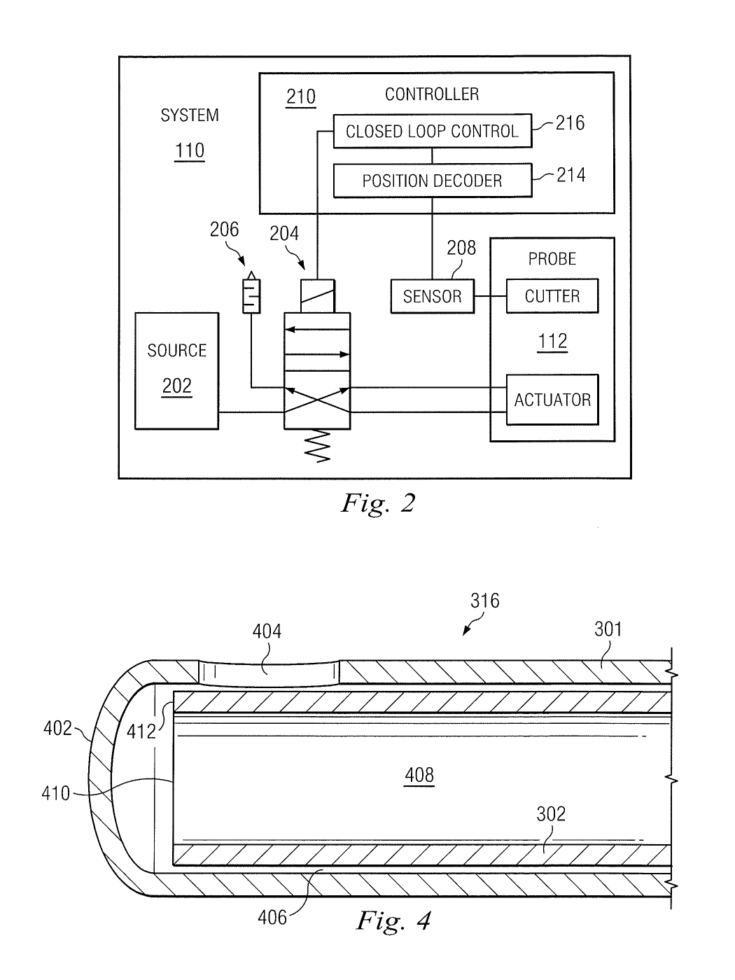

FIG. 2 is a schematic of the vitrectomy probe system 110 that provides closed-loop position control according to one exemplary embodiment. In FIG. 2, the probe system 110 includes the vitrectomy probe 112, a pneumatic pressure source 202, a probe driver shown as an adjustable directional on-off pneumatic driver 204, a muffler 206, a sensor 208, and a controller 210. As can be seen, the source 202, the driver 204, the muffler 206, and the probe 112 are in fluid communication with each other along lines representing flow paths or flow lines. The controller 210 is in electrical communication with the driver 204 and the sensor 208.

Although the sensor 208 is shown separate from the probe 112 in FIG. 2, the sensor 208 is arranged and positioned to sense data from the probe 112. Accordingly, in some embodiments, instead of being outside of the probe 112, it may be disposed internal of the probe, as is shown in the example in FIG. 3, described below.

FIG. 3 shows a cross-sectional illustration of an exemplary vitrectomy probe, referenced by the numeral 112. In this example, the vitrectomy probe 112 is a pneumatically driven probe that operates by receiving pneumatic pressure alternating through first and second ports 312 and 314. The probe 112 includes as its basic components a cutter 300 comprising an outer cutting tube 301, an inner cutting tube 302, and a probe actuator or motor shown here as a reciprocating air driven diaphragm 304, all partially encased by a housing 306. The housing 306 includes an end piece 308 at the probe proximal end with the first and second air supply ports 312, 314 and one suction port 310. The sensor 208 is disposed within the probe housing 306 at a location to monitor the relative displacement of the outer and inner cutting tubes 301 and 302.

As can be seen, the cutter 300 extends from the housing 306 and includes a distal end 316. FIG. 3 shows the distal end 316 of the cutting tube 300 in greater detail. The cutter 300 includes the outer cutting tube 301 that has a closed end 402, and an outer port 404 that receives tissue, such as ophthalmic tissue. The outer port 404 is in fluid communication with an inner channel 406 of the outer cutting tube 301. The inner cutting tube 302 is located within the inner channel 406 of the outer cutting tube 301. The inner cutting tube 302 has an inner bore 408, an open end 410, and a cutting surface 412. The inner bore 408 is in fluid communication with an aspiration line (not shown) that connects to a vacuum pressure that pulls tissue into the outer port 404 when the inner cutting member 302 is located away from the port 404. The inner cutting tube 302 moves within the inner channel 406 of the outer cutting tube 301 to cut tissue that is pulled into the outer port 404 by the aspiration system. The ophthalmic tissue received by the outer port 404 is preferably vitreous or membranes.

When used to cut tissue, the inner cutting tube 302 is initially moved away from the outer port 404 and the vacuum pressure pulls tissue into the port 404 and the inner channel 408. The inner cutting tube 302 then moves toward the outer port 404 and severs the tissue within the inner channel 406. The severed tissue is pulled through the inner bore 408 of the inner cutting tube 302 by the aspiration system. The inner cutting tube 302 then moves away from the outer port 404, and the cutting process is repeated. A cutting cycle includes moving the inner cutting tube 302 to open the port 404 and then moving the cutting tube 302 to close the port 404 to initiate the cut and return the cutting tube 302 to its starting position for the next cutting cycle.

The actuation of the inner cutting tube 302 opens the port 404 for a fixed amount of time in each cut cycle of the probe 100. In some embodiments, for a given vacuum level or a given flow rate, this results in a relatively consistent volume of cut ophthalmic tissue regardless of the probe cut rates. The amount of time the port 404 is open in each cut cycle is, in some examples, about 1.5 milliseconds to about 2.5 milliseconds.

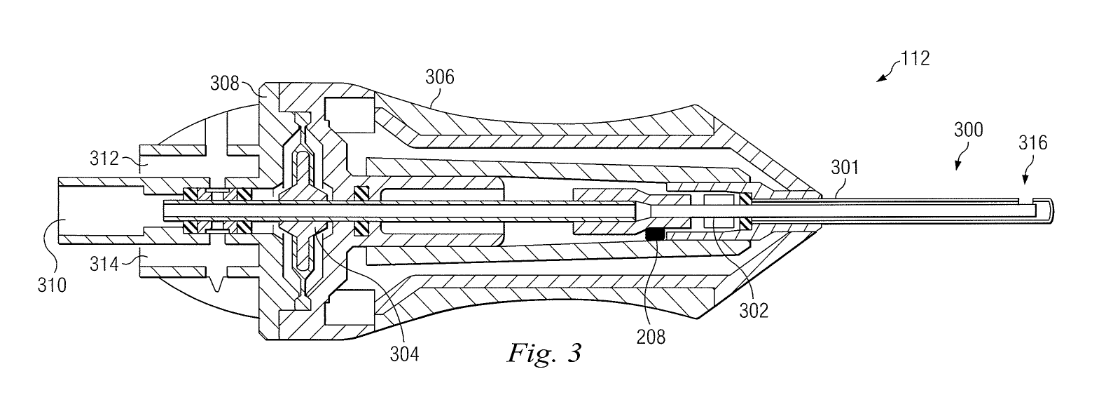

With reference now to both FIGS. 3 and 4, the inner cutting tube 302 is driven by air pressure directed on opposing sides of the diaphragm 304. In one example of operation, if air pressure is increased at the first port 312, the diaphragm 304 will move distally, displacing the inner cutting tube 302 relative to the outer cutting tube 301, thereby closing the tissue-receiving port 404 of the outer cutting tube 301. This cuts any vitreous material which may have been aspirated into the tissue-receiving outer port 404. Venting the pressure at the first port 312 and increasing the pressure at the second port 214 will move the diaphragm 304 proximally, opening the tissue-receiving outer port 404 so that it can draw in new vitreous material to be cut. Its worth noting that other embodiments include alternative probe actuators. For example, some embodiments, include a piston motor in place of a diaphragm. In this type of embodiment, the cutter 300 is arranged so that movement of the piston also moves the inner cutting tube 302 of the cutter 300. Yet other embodiments include other types of pneumatic or electric motors that drive the inner cutting tube 302.

Returning to FIG. 2, in the example shown, the vitrectomy probe system's pneumatic driver 204, is a standard four-way on-off valve. As is commonly known, the pneumatic driver 204 has a solenoid that operates to move the driver to one of the two on-off positions depicted in the example of FIG. 2. Here, the pneumatic driver 204 is in a position to provide pneumatic pressure to the first port 312, and to vent pneumatic pressure from the second port 314. In this position, pneumatic pressure can pass from the pressure source 202, through the on-off pneumatic driver 204, and to the first port 312 where the pneumatic pressure provides pneumatic power to the vitrectomy probe. At the same time, pneumatic pressure at the second port 314 can pass through the on-off pneumatic driver 204 to the muffler 206 where it is exhausted to the atmosphere. In the other position, the on-off pneumatic driver 204 allows pneumatic pressure to pass from the pressure source 202 to the second port 314 where the pneumatic pressure provides pneumatic power to the vitrectomy probe 112. At the same time, pneumatic pressure at the first port 312 can vent through the on-off pneumatic driver 204 to the muffler 206 where it is exhausted to the atmosphere. The on-off pneumatic driver is configured to receive operating signals from the controller 210 as further described below.

In operation, pneumatic pressure is directed alternately from the source 202 to the first and second ports 312, 314 to operate the vitrectomy probe 112. The on-off pneumatic driver 204 alternates between its two positions very rapidly to alternatingly provide pneumatic pressure to the first and second ports 312, 314.

Although shown with a single pneumatic driver 204, other embodiments include two pneumatic drivers, one associated with each of the two ports 312, 314. These embodiments operate similar to the manner described, with the drivers being configured to independently receive operating signals from the controller 210. Yet other arrangements are contemplated.

The sensor 208 is disposed in a location to monitor displacement of the inner cutting tube 302 relative to the outer cutting tube 301. In some examples, the sensor 208 is disposed in the interior of the housing 306, while in other embodiments, it is disposed exterior of the housing 306. In yet other embodiments, the sensor 208 is disposed to monitor displacement of the cutter assembly, and not just the cutter itself. The cutter assembly may include components configured to drive the inner cutting tube 302 of the cutter 300. For example, in some embodiments, the sensor 208 may be configured to monitor displacement of a diaphragm motor fixed to the inner cutting tube 302. In yet other embodiments, the sensor 208 may be disposed to monitor displacement of the diaphragm 304 driving the inner cutting member 302. As used herein, monitoring the displacement of the inner cutting tube is intended to encompass both direct monitoring, such as monitoring the cutter itself, and indirect monitoring, such as monitoring a motor that drives the inner cutting tube. The sensor may be comprised of any type of sensor suitable for measuring a physical displacement. For example, the sensor may be a fiber optic sensor, a linear variable differential transducer (LVDT), a power spectral density (PSD) laser, a change couple device (CCD) laser, or other sensor.

Based upon signals receive and generated by the sensor 208, the controller 210 can monitor and adjust the stroke of the inner cutting member 302 of the cutter 300 by varying the servo output to correct and optimize the cutter action using a closed loop feedback process.

The controller 210 comprises a processor and a memory and is configured to receive data, perform functions, and execute programs stored in the memory. In different embodiments, the controller 210 is, for example, a PID controller, an integrated circuit configured to perform logic functions, or a microprocessor that performs logic functions. It may include a memory and a processor that may execute programs stored in the memory. In some embodiments, the memory stores stroke length data, frequency data, and port size data, particular desired time lengths, and desired stroke lengths, among other parameters, for particular duty cycles or cut rates of the vitrectomy probe 112. Memory of the controller 210 is typically a semiconductor memory such as RAM, FRAM, or flash memory. The memory interfaces with the processor. As such, the processor can write to and read from the memory. In this manner, a series of executable programs can be stored in the memory. The processor is also capable of performing other basic memory functions, such as erasing or overwriting the memory, detecting when the memory is full, and other common functions associated with managing semiconductor memory.

The controller 210 includes a position decoder 214 and a closed loop control module 216. The position decoder 214 is arranged and structurally configured to receive an analog from the sensor 208 and filter, interpret, or digitize the signal for processing by the closed loop control module. The closed loop control module 216 then receives the signal from the position decoder 214 and processes it according to a stored instructions to provide a real time assessment of whether the vitrectomy probe is operating in the manner desired, and then is configured to control operation of the pneumatic driver 204 based on the feedback received from the sensor 208.

In some embodiments, the controller 210 is configured to receive signals from the sensor 208 representative of the position of the inner cutter tube and from that, calculate and control the pneumatic driver 204 to maximize cutting speed change probe duty cycle (as opposed to valve duty cycle) to avoid wasted energy and excessive motion.

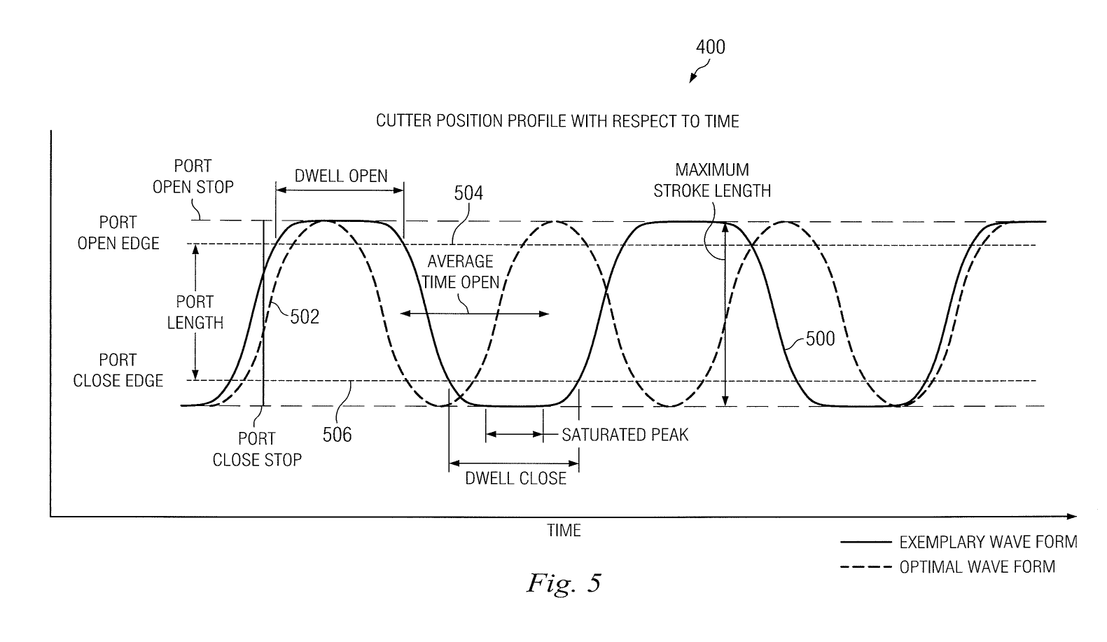

FIG. 5 shows an exemplary wave form 500 representing a signal from the sensor 208 indicating axial displacement of the inner cutting tube 302 relative to the outer cutting tube 301 during operation of the probe 112. It also shows an exemplary ideal wave form 502 representing a target or optimal waveform according to one exemplary desired aspect. As shown in FIG. 5, the output signal 500 is a sinusoidal type of waveform. The amplitude 504 represents the distance the inner cutting tube 302 travels to its open position, fully opening the port 404. Similarly the amplitude 506 represents the distance the inner cutting tube 302 travels to its closed position. The peak to peak amplitude differential is the maximum stroke length of the cutter. A saturated peak amplitude (i.e. when the cutter is dwelling in the closed or open state), indicates the cutter's maximum closed or minimum open positions. That is, the saturated peak amplitude represents a hard mechanical stop that prevents further displacement beyond the maximum closed or minimum open position, resulting in an idle or stopped inner cutting tube.

The probe actuator, whether a diaphragm, piston, or other motor, must drive the cutter 300 to fully close the port 404, but if desired, need not fully open the port. A preferred stroke length is one that meets or exceeds the preferred cutter position profile in FIG. 5 that first opens the port 404 to receive tissue through the port 404 and second fully advances the inner cutting 302 to a location sufficient to close the port 404 to cut the tissue that entered the port.

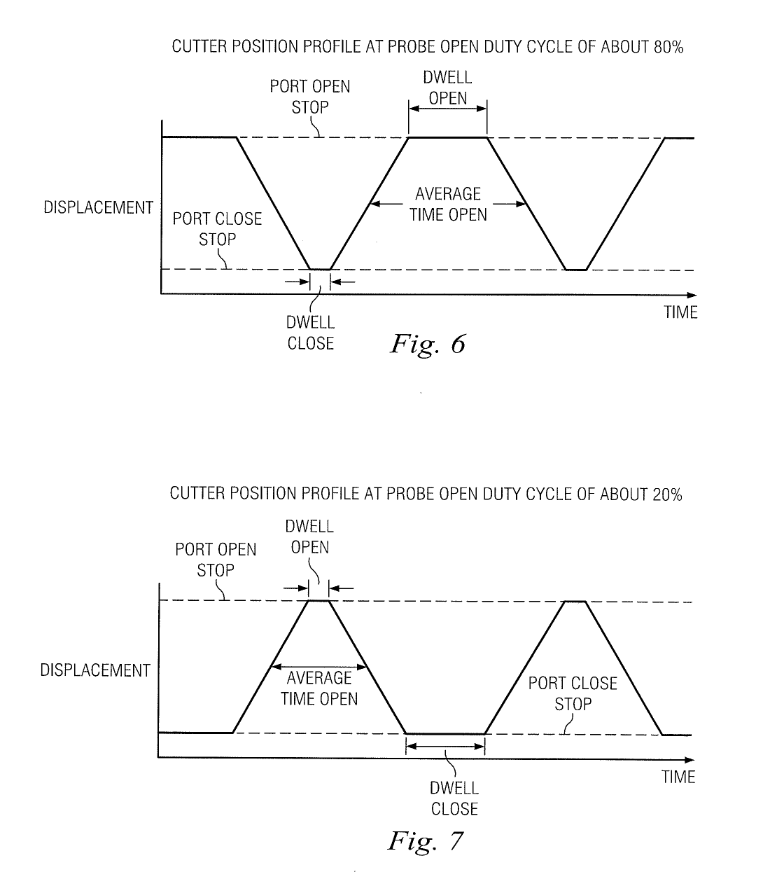

The dashed line 502 in FIG. 5 represents an exemplary desired or optimized stroke, with a stroke length having minimized dwell times and without any saturated peak. This optimized stroke permits higher cut rates than can be achieved by general signals having saturated peaks and long dwell times. The system disclosed herein enables a user to control a specific cutter to achieve the desired cutting profile to optimize cutting. In this example, the desired stroke has a length designed to minimize the actual stroke length, while still fully closing and fully opening the port 404. In some examples, if desired, the desired stroke length may be a stroke that fully closes the port 404, but only partially opens the port 404. In yet other examples, the desired stroke length may be one that includes a duty cycle with dwell times in the open and closed positions, depending on the application and the surgeon's preferences. The exemplary stroke 502 is shown with a 50% duty cycle. Other examples have a desired stroke with a varied duty cycle that increases or decreases the dwell times in the close position or open position, as shown in FIGS. 6 and 7.

FIGS. 6 and 7 show exemplary wave forms representing cutter position profiles with probe duty cycles of 80% and 20% respectively. Referring first to FIG. 6, a probe open duty cycle of 80% results a long dwell time in the open position and a short dwell time in the closed position, with a relatively lengthy average time in the open position. FIG. 7, in contrast, shows a probe open duty cycle of 20% resulting a short dwell time in the open position and a longer dwell time in the closed position, with a relatively short average time in the open position. Since the actual relative position of the inner cutting tube 302 is determined using the sensor 208, the controller 210 can adjust the or control the valve 204 to achieve any desired duty cycle or to operate at an optimum profile that enables higher cut rates than can be consistently achieved in conventional systems. In conventional open loop systems, variations in actuator characteristics due to initial tolerances or degradation and wear over time could potentially degrade the performance of the actuator, prevent the actuator from fully opening or fully closing, or result in excessive stroke length or excessive saturated peak times, leading to wasted energy and artificial limits on cutter speed.

However, in the present system, the controller 210 is configured to compensate for component tolerances and variations by receiving signals from the sensor 208 and measuring and tracking the position of the inner cutting tube 302 relative to the outer cutting tube 301 to achieve a desired cutter position profile that maximizes cut rate or changes probe duty cycle. The probe duty cycle is the ratio of time that the cutting tubes is open to the time that the cutter is closed. By detecting and tracking the actual position of the inner and outer cutting tubes 301, 302, the controller 210 may modify the control signals sent to the probe driver shown as the on-off pneumatic driver 204 to adjust the probe's duty cycle or stroke length or other parameter that will result in increased efficiency. It can do this based on control laws that determine whether adjustments should be made to signals being sent to the on-off pneumatic driver 204. This becomes more clear with reference to an exemplary method below of using feedback control for the vitrectomy probe.

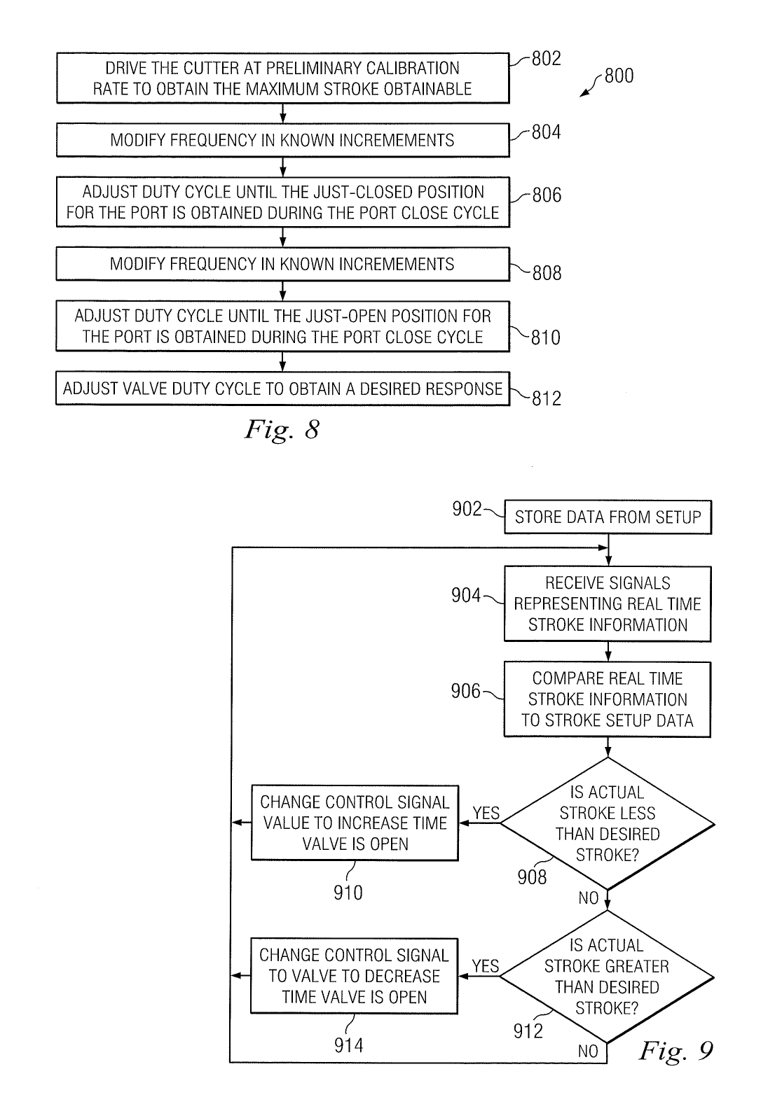

FIG. 8 shows an exemplary control loop 800 for generating and using position feedback control to increase efficiency and overcome component variation, such as may occur with, for example, tolerance build up or wear. An exemplary method of feedback control will be described with reference to the control loop 800.

Prior to full operation some embodiments employ a relatively short calibration cycle or setup cycle to determine information about the particular cutter, such as the maximum stroke of the cutter 300 and the timing required to obtain desired strokes. In some examples, the method is performed as a part of an initial start-up routine when components of the probe are changed or modified. In other examples, the method is performed each time the probe is activated after a period of inactivity, even if components are not replaced or modified.

One example of the setup cycle performed by the system includes operating the pneumatic driver 204 (FIG. 2) to drive the cutter 300 at a known preliminary cut rate, such as, for example, 1000 cpm to obtain the maximum stroke length, as at step 802. To do this, the controller 210 generates and sends control signals to the pneumatic driver 204 to control the frequency and pneumatic driver duty cycle to ensure the maximum stroke of the cutter is attained. The on-off switching of the pneumatic driver 204 results in an oscillating pressure signal through the lines connecting the pneumatic driver 204 to the probe 112. These oscillating pressures drive the probe actuator (shown as the diaphragm 304 in FIG. 3) in an oscillating manner. The probe actuator 304 likewise drives the inner cutting tube 302 of the cutter 300. As described above, the sensor 208 monitors the displacement of the inner cutting tube 302 relative to the outer cutting tube 301 either directly or indirectly and outputs a signal indicative of the position of the inner and outer cutting tubes 301, 302. This signal is communicated from the sensor 208 to the position decoder 214 in the controller 210. This signal is represented in FIG. 5 and is an analog signal varying from a maximum to a minimum sensor voltage, with saturated peaks that relate to the maximum closed and open positions of the cutter.

Once the maximum stroke is known based on the saturated peaks, the controller 210 generates and outputs signals to the pneumatic driver 204 to operate for a period of time at a number of different known frequencies, as at step 804. In one example, the controller 210 sends signals to the pneumatic driver 204 to sweep from low to high frequency in incremental steps. For example, the pneumatic driver 204 may be controlled to operate from low to high frequency, such as, for example, from 1000 to 10,000 cpm in 1000 cpm increments. At each increment, the duty cycle of the pneumatic driver 204 is adjusted from low to high to obtain data representative of a stroke length that is shorter than the maximum stroke length, and that coincides with a just-closed position of the cutter, as indicated at step 806. This “just closed” position is the position of the inner cutting tube 302 relative to the outer cutting tube where the port 404 is sufficiently closed so that tissue is cut, but the inner cutting tube moves only slightly beyond the port, so that energy is not wasted and the travel length of the inner cutting tube is sufficient but minimized. This is represented in FIG. 5 by the dashed sinusoidal line below the “Port Close Edge” boundary.

This just-closed position is then stored in the controller 210 as a desired closed position. The travel to the desired closed position may be later tracked using the analog signal from the sensor 208, as shown in FIG. 5.

Once the desired closed position is determined, along with the desired signal from the sensor, at a step 808 the probe is driven again at the same low and high frequencies (i.e., in the example above, from 1000 to 10,000 cpm in 1000 cpm increments). At each frequency increment, the duty cycle of the valve or other driver is adjusted from low to high to obtain data representative of a stroke length that is shorter than the maximum stroke length, and that coincides with a just-open position of the cutter, as at a step 810. In some examples, the just-open position is the position of the inner cutting tube 302 relative to the outer cutting tube where the port 404 is opened a desirable amount to permit tissue to enter and be cut as desired during a ophthalmic surgical procedure. Accordingly, in some examples, the just-open position is the position of the inner cutting tube 302 relative to the outer cutting tube where the port 404 is fully-opened to receive the maximum amount of tissue, but the inner cutting tube moves only slightly beyond the port, so that energy is not wasted and the travel length of the inner cutting tube is sufficient but minimized. This is represented in FIG. 5 by the dashed sinusoidal line above the “Port Open Edge” boundary.

This just-open position at each frequency is then stored in the controller as a desired open position. The travel to the open position corresponds to the analog signal from the sensor 208, as shown in FIG. 5.

With the operating duty cycle profiles established and stored for continued access by the controller 210, the controller 210 can rely on this data to adjust the pneumatic driver duty cycles to obtain a desired response from the probe (i.e. desired port duty cycle or variable position of the cutter), as at step 812.

The controller 210 stores all the cutter information with stroke lengths at particular frequencies and duty cycles. This information is then available for use as reference settings during normal operation of the cutter 300. Accordingly, the controller 210 is configured to compare actual detected measurements to those reference settings obtained during the setup phase, and adjust the actual settings based on the comparison so that the actual settings correspond to the reference settings. This is described with reference to FIG. 9 below.

In use, the system 110 receives an input from a health care provider setting a particular cut rate and/or duty cycle. This may be done using an input device on the machine 100 or on the vitrectomy probe 112. Input examples may include squeezing the probe handle to adjust the duty cycle and inputting via selection on a screen using a keyboard, mouse, knobs, or other known input device. In some examples, the operating settings are prestored in the system using default or pre-programmed values. The system then initializes and operates at that particular setting to provide the desired cutting parameter.

Referring to FIG. 9, at a step 902 and as described above, the controller 210 stores data from the setup sequence. At a step 904, during normal operation, the controller 210 receives data from the sensor 208 regarding the actual real-time position of the cutting mechanism 300. The analog signal from the sensor 208 may be filtered, interpreted, or digitized by the position decoder 214 prior to processing by the closed loop control module 216 in a manner known in the art to provide meaningful data for treatment by the controller 210.

At a step 906, the closed loop control module 216 compares the real-time stroke information to the stored reference data obtained during the setup sequence. Through this, the controller 210 is able to determine whether the cutter 300 should be adjusted to optimize the stroke and obtain the desired cutting characteristics, including for example, dwell times and stroke lengths.

At a step 908, the closed loop control module 216 determines whether the actual stroke is less than the desired stroke. If the actual stroke is less than the desired stroke the closed loop control module 216 adjusts the control signal sent to the probe driver 204 to modify the actual stroke to more closely correspond to the desired stroke. For example, if the actual stroke is less than the desired stroke at step 908, then the closed loop control module 216 changes the control signal to increase the time that the pneumatic driver is open in each position at a step 910, thereby increasing the time period that the cutter travels in one direction. This modifies the operational timing of the driver and effectively increases the stroke length of the cutter 300. In addition, depending on the desired duty cycle, step 910 may include controlling the driver 204 to change the stroke timing so that it corresponds to the desired stroke.

If at step 908, the actual stroke is not less than the desired stroke, then the controller moves to a step 912 and determines whether the actual stroke is greater than the desired stroke. If at step 912, the controller 210 determines whether the actual stroke is greater than the desired stroke, the closed loop control module 216 adjusts the control signal sent to the probe driver 204 to modify the actual stroke to more closely correspond to the desired stroke. For example, if the actual stroke is greater than the desired stroke at step 912, then the closed loop control module changes the control signal to decrease the time that the pneumatic driver is open in each position at a step 914, thereby decreasing the time period that the cutter travels in one direction. In addition, as indicated above, depending on the desired duty cycle, step 914 may include controlling the driver 204 to change the stroke timing and the duty cycle of the cutter so that it corresponds to the desired stroke. This modifies the operational timing of the driver and effectively decreases the stroke length of the cutter 300. If at step 912, the actual stroke is not greater than the desired stroke, then the controller returns to step 904, and again receives the signals representing the real time stroke information.

Accordingly, instead of operating a vitrectomy probe according to parameters suitable for a large population of probes, the methods and systems disclosed herein operate a vitrectomy probe according to personalized parameters ideal just for the particular probe. In addition, by implementing a closed loop control system for the vitrectomy probe, the probe's cutter movement can be optimized to maximize the full performance of the system and increase the robustness of the design. Thus, current probes can be used at increased cutting rates, and/or probe designs can be made easier to build while still obtaining similar or better performance than current probes.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I claim:

1. A surgical system having position feedback control, comprising:
a probe driver;
a vitrectomy probe having a cutting mechanism, the cutting mechanism having an inner cutting tube and an outer cutting tube, the outer cutting tube having a tissue-receiving port formed therein, the inner cutting tube having a cutting edge axially displaceable relative the tissue receiving port to cut tissue therein;
a sensor disposed and configured to detect the displacement of the inner cutting tube relative to the outer cutting tube and communicate a signal indicative of the relative displacement of the inner cutting tube; and
a controller in communication with the sensor and with the probe driver, the controller being configured to change operational timing of the probe driver based on the signal communicated from the sensor.

2. The surgical system of claim 1, wherein the controller is configured to determine a desired stroke for the inner cutting tube based on the signal communicated from the sensor.

3. The surgical system of claim 2, wherein the desired stroke is a stroke length designed to minimize the stroke length while providing suitable tissue cutting for a procedure.

4. The surgical system of claim 3, wherein the controller is configured to control the probe driver based on the signal communicated from the sensor to increase or decrease the relative displacement to comply with the desired stroke.

5. The surgical system of claim 4, wherein the controller is configured to control the probe driver by changing the duty cycle.

6. The surgical system of claim 2, wherein the desired stroke length is less than the maximum possible stroke length.

7. The surgical system of claim 1, wherein the vitrectomy probe is a pneumatically driven vitrectomy probe.

8. The surgical system of claim 1, wherein the controller comprises a position decoder configured to interpret analog signals received from the sensor.

9. The surgical system of claim 1, wherein the controller is configured to determine the just-closed and just-fully open positions of inner cutting tube relative to the port on the outer cutting tube.

10. The surgical system of claim 1, wherein the controller is configured to compare the sensed relative displacement of the inner cutting tube to a desired displacement and modify the operational timing of the valve based on the difference between the sensed relative displacement and the desired displacement.

11. A method of controlling a surgical system using position feedback control, comprising:
selectively operating a cutter having a cutting mechanism, the cutting mechanism having an inner cutting tube and an outer cutting tube, the outer cutting tube having a tissue-receiving port formed therein, the inner cutting tube having a cutting edge axially displaceable relative the tissue receiving port to cut tissue therein;
sensing the displacement of the inner cutting tube relative to the outer cutting tube with a sensor; and
changing operational timing of a probe driver based on the displacement sensed by the sensor.

12. The method of claim 11, comprising comparing a real time stroke of the inner cutting tube to a desired stroke length to optimize a stroke length of the cutter.

13. The method of claim 12, comprising tracking the real time stroke length of the cutter, and comparing the stroke length to a stored stroke length.

14. The method of claim 11, comprising determining whether the actual stroke length of the cutter as detected by the sensor is greater than the desired stroke length.

15. The method of claim 14, comprising determining whether the actual stroke length of the cutter is less than the desired stroke length.

16. The method of claim 11, wherein changing the operational timing of the probe driver includes modifying a control signal to a valve to operate the cutter in a different manner.

17. The method of claim 11, comprising:
developing a reference data set having optimized target cutter data; and
comparing the sensed displacement of the inner cutting relative to the outer cutting tube to the optimized target cutter data.

18. The method of claim 15, wherein developing a reference data set comprises operating the cutter mechanism at a known cut rate and identifying the maximum stroke length of the cutter.

18. The method of claim 15, wherein developing a reference data set comprises operating the cutter mechanism at a known cut rate and determining when the inner cutting mechanism reaches a just-closed position.

19. The method of claim 18, wherein developing a reference data set comprises operating the cutter mechanism at a known cut rate and determining when the inner cutting mechanism reaches the just open position.

20. A method of using position feedback control to control a cutting mechanism of a vitrectomy probe having an inner cutting tube and an outer cutting tube, the outer cutting tube having a tissue-receiving port formed therein the inner cutting tube having a cutting edge axially displaceable relative the tissue receiving port to cut tissue therein, the method comprising:
setting up the vitrectomy probe to detect a port just open position and a port just closed position;
identifying a target stroke length from the port just open position to the port just fully closed position;
receiving an input from a health care provider during a surgical procedure;
sensing an actual stroke length during the surgical procedure with a sensor;
comparing the actual stroke length to the target stroke length; and
adjusting the system to change the actual stroke length to more closely match the target stroke length.

21. The method of claim 20, wherein setting up the vitrectomy probe comprises operating the cutter mechanism at a known cut rate and determining when the inner cutting mechanism reaches the just closed position.

22. The method of claim 21, wherein calibrating the vitrectomy probe comprises operating the cutter mechanism at a known cut rate and determining when the inner cutting mechanism reaches the just-fully open position.

23. The method of claim 20, wherein sensing the actual length includes sensing the travel to the closed position and travel to the open position using an analog sinusoidal wave.
